(19) United States
(12) Patent Application Publication
(10) Pub. No.: US 2026/0294374 A1
(43) Pub. Date: Oct. 1, 2026

(54) Image Processing Method

(71) Applicant: Mako Surgical Corp., Fort Lauderdale, FL (US)

(72) Inventor: Michael Antony Bowes, Manchester (GB)

(21) Appl. No.: 19/489,409

(22) PCT Filed: May 31, 2024
(86) PCT No.: PCT/GB2024/051427

§ 371 Date: Dec. 2, 2025

(30) Foreign Application Priority Data
Jun. 2, 2023 (GB) 2308267.0

(51) Int. Cl. A61B 6/50; A61B 6/00; G01R 33/56; G06T 7/00

(52) U.S. Cl. CPC A61B 6/505; A61B 6/5217; G01R 33/5608; G06T 7/0012; G06T 2207/10081; G06T 2207/10088

(57) ABSTRACT

A method of determining a property of cartilage in a joint of interest of a patient. The method comprises receiving data indicating joint space width of the joint of interest, receiving data indicating a relationship between joint space width and a property of cartilage in the joint of interest, wherein the data indicating a relationship between joint space width and the property of cartilage in the joint of interest is based upon measured cartilage in a population of joints of interest, and determining the property of cartilage in the joint of interest based upon the data indicating joint space width of the joint of interest and the data indicating a relationship between joint space width and property of cartilage in the joint of interest.

### BACKGROUND

The present invention relates to methods and apparatus for predicting a state of a musculoskeletal joint. More particularly, the present invention relates to methods and apparatus for determining a property of cartilage in a joint of interest such as an ankle joint, a knee joint, a hip joint, a shoulder joint, a spine joint, or an elbow joint.

With ageing populations degenerative joint diseases have become common place, with around 8 million people in the UK and around 27 million people in the US suffering from osteoarthritis.

The causes of osteoarthritis, as well as some other degenerative joint diseases, are not well understood, however, a number of factors are believed to contribute to likelihood of disease onset in certain joints such as the knee. Such factors include high body mass index, traumatic damage to ligaments and menisci, valgus alignment of the knees, and the way in which the joint is used in daily activities.

All of the tissues in the knee may be affected by degenerative joint disease, including bone, cartilage, menisci, ligaments, capsule and synovial tissue. Individual patients may have damage to some or all of these tissues, and at a particular point in time, each patient knee may contain any combination of damaged tissues.

Diagnosis of osteoarthritis typically is by radiographic scoring of bone change (osteophytes and bone damage) and joint space narrowing (indicating that cartilage has been lost from the surfaces of articulating bone). For example, the Kellgren and Lawrence classification system grades osteoarthritis into one of five grades, with each grade being based upon a combination of joint space narrowing, presence or absence of osteophytes, bone damage and their respective severity.

Various medical and surgical interventions for degenerative joint diseases exist. For example, modern orthopaedic surgery allows routine surgical repair of joints, sometimes referred to as arthroplasty. Arthroplasty may involve reshaping of a joint by modifying an existing joint surface and bone to provide a joint that operates more effectively. In recent decades, surgical replacement of joints or joint surfaces with a prosthesis has been the most common and successful form of arthroplasty and has become a common treatment for osteoarthritis. Whether such medical and surgical interventions are performed is typically based at least in part upon diagnosis by radiographic scoring of bone change and joint space narrowing.

There remains, however, a need for improvements in providing information of a state of joints of interest for clinicians for diagnosing and treating degenerative joint diseases such as osteoarthritis.

### SUMMARY

There is described herein a method of determining a property of cartilage in a joint of interest of a patient. The method comprises receiving data indicating joint space width of the joint of interest, receiving data indicating a relationship between joint space width and the property of cartilage loss in the joint of interest, wherein the data indicating a relationship between joint space width and the property of cartilage loss in the joint of interest is based upon measured cartilage loss in a population of joints of interest, and determining the property of cartilage in the joint of interest based upon the data indicating joint space width of the joint of interest and the data indicating a relationship between joint space width and the property of cartilage loss in the joint of interest.

By determining the property of cartilage in the joint of interest according to the methods described herein, imaging properties of cartilage at a given point in time using often complex, expensive, and time consuming methods can be avoided while still providing useful diagnostic information relating to cartilage and patient outcomes can be improved.

In some implementations, the property of cartilage loss is a likelihood of cartilage loss or a structural quality of cartilage. By determining a property of cartilage that is a likelihood of cartilage loss, not only may an indication that the joint of interest has lost more than a usual amount of cartilage be determined, but a determination that the joint of interest will be likely to continue to lose cartilage at a higher than usual rate may also be determined. By determining a property of cartilage that is a structural quality of cartilage, the joint of interest may be assessed accordingly.

In some implementations, the data indicating joint space width of the joint of interest comprises one or more normalized values associated with the joint space width. By using one or more normalized values, variation and deviation from an average may be more easily quantified.

In some implementations, each patient associated with the one or more healthy joints do not exhibit symptoms associated with a disease affecting cartilage. For example, the patients may be selected based upon one or more of age, whether the patient has pain in the joint of interest or any other property associated with disease. By selecting patients associated with healthy joints only it has been determined that a normal distribution of properties is observed. Such a distribution of properties allows determination that a value falls outside of the normal distribution to be accurately classified as indicative of a particular property such as cartilage loss.

In some implementations, the one or more normalized values are determined based upon the sex of the patient. By normalizing based upon the sex of the patient, significant differences in the average joint space width and/or cartilage thickness between males and females may be accounted for. It has been surprisingly realised that normalizing based upon sex allows ally significant variation to be accounted for. That is, it has been determined that it is only necessary to normalize based upon the sex of the patient and that no other patient-specific properties need to be accounted for in the normalization to provide a single value representative of cartilage thickness.

In some implementations, the one or more normalized values are determined based upon healthy joint space width data indicating healthy joint space width for one or more healthy joints.

In some implementations, the one or more normalized values indicate a variation from one or more average values associated with one or more healthy joints corresponding to the joint of interest. In some implementations, the average value is determined based upon sex. The normalized values may provide a standard score that allows patients to be compared. The standard score may, for example, be a z-score.

In some implementations, the data indicating joint space width is determined based upon one or more images of the joint of interest. In some implementations, the data indicating joint space width of the joint of interest is determined based upon image processing that does not determine cartilage. In some implementations, the one or more images are generated by computed tomography and/or magnetic resonance imaging. For example, the data indicating joint space width may be determined from an imaging modality in which it is not normally possible to identify cartilage, or in which additional image processing is required to identify cartilage. The imaging modality may be such that cartilage cannot be accurately measured.

In some implementations, the data indicating joint space width of the joint of interest is determined from first image data and the data indicating a relationship between joint space width and the property of cartilage in the joint of interest is determined from second image data different to the first image data. The data indicating joint space width may be determined from image data obtained from a different imaging modality to the imaging modality used to obtain patient data used for generating the model.

In some implementations, the data indicating a relationship between joint space width and the property of cartilage in the joint of interest is obtained based upon data obtained from a plurality of joints of interest in which joint space width and the property of cartilage in the joint of interest are known. The plurality of joints of interest may be from individuals other than the patient. In this way, data indicating a property of cartilage in a population may be used to predict the property of cartilage in an individual.

In some implementations, the joint of interest comprises a lateral compartment and a medial compartment. In some implementations, the data indicating joint space width comprises lateral joint space width data corresponding to the lateral compartment and/or medial joint space width data corresponding to the medial compartment. Both lateral and medial compartments may additionally or alternatively be considered in combination.

In some implementations, the method described herein may further comprise determining, based upon the property of cartilage, whether or not the joint of interest is affected by a degenerative joint disease such as osteoarthritis.

In some implementations, the data indicating joint space width is determined based upon a plurality of measurements between predetermined points of the joint of interest. In some implementations, the data indicating joint space width comprises an average of the plurality of measurements, a centile of the plurality of measurements, and/or median of the plurality of measurements. The centile may be determined based upon a portion of bones of the joint of interest that are adjacent to one another in an imaged state.

In some implementations, the joint of interest is an ankle joint, a knee joint, a hip joint, a shoulder joint, a spine joint, or an elbow joint. In some implementations, the data the indicating joint space width of the joint of interest is associated with one or more of the tibia of the knee joint, the fibia of the knee joint and the patella of the knee joint. Lateral and medial compartments may additionally be considered.

In some implementations, the property of cartilage in the joint of interest may be used to select and/or generate a prosthesis for the patient. The property of cartilage in the joint of interest may additionally or alternatively be used to determine a surgical intervention and/or surgical plan for the patient.

In some implementations, the data indicating joint space width is generated based upon joints of interest under compression and/or load bearing. For example, the joints of interest may be imaged when the joint is under compression and/or load bearing. By determining the data indicating joint space width for joints of interest under compression, any mechanical or positional factors which may affect a true indication of joint space width may be mitigated.

There is also described herein a computer program comprising computer readable instructions as set out above. There is also described herein a computer readable medium carrying a computer program as set out above. There is also described herein a computer apparatus for determining a property of cartilage in a joint of interest of a patient, the apparatus comprising a memory storing processor readable instructions, and a processor arranged to read and execute instructions stored in memory, the said processor readable instructions comprising instructions as set out above.

While specific implementations have been described above, it will be appreciated that the invention may be practiced otherwise than as described. The descriptions above are intended to be illustrative, not limiting. Thus it will be apparent to one skilled in the art that modifications may be made to the invention as described without departing from the spirit of the invention.

### BRIEF DESCRIPTION OF THE FIGURES

FIG. 1 depicts a system for generating anatomical data providing a likelihood of cartilage loss in a joint of interest of a patient.

FIG. 2 depicts a computer system in which the techniques described herein may be implemented.

FIG. 3 depicts a flow diagram of a method for generating anatomical data providing a likelihood of cartilage loss in a joint of interest of a patient.

FIG. 4a depicts a knee joint, its respective compartments, and patches used to measure joint space width.

FIG. 4b depicts masks used for shape modelling a tibia.

FIG. 5a depicts two normal distributions for medial joint space width, one for male patients and another for female patients.

FIG. 5b depicts two normal distributions for lateral joint space width, one for male patients and another for female patients.

FIG. 6 depicts a system used to generate relationship data indicating a relationship between joint space width and likelihood of cartilage loss.

FIG. 7 depicts a flow diagram of a method for determining relationship data.

FIG. 8 depicts a scatter plot of lateral and medial joint space widths and their corresponding indication of a likelihood of cartilage loss.

FIG. 9 depicts a first relationship between ranges of values indicative of joint space width and a corresponding likelihood of cartilage loss.

FIG. 10 depicts a second relationship between ranges of values indicative of joint space width and a corresponding likelihood of cartilage loss.

FIG. 11 depicts a knee joint, its respective compartments, and a corresponding likelihood of cartilage loss for each compartment.

Reference numerals that are repeated across plural figures are intended to identify the same features in various implementations.

### DETAILED DESCRIPTION

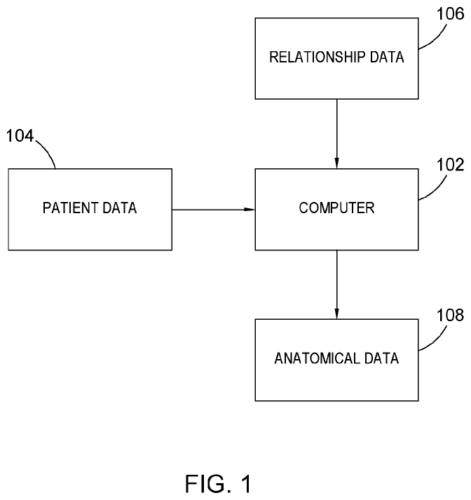

Referring to FIG. 1, a computer 102 is arranged to receive patient data 104 indicating a joint space width associated with a musculoskeletal joint of interest of a patient. For example, the musculoskeletal joint of interest of the patient may be a knee joint or an ankle joint. It will be readily appreciated that the methods and systems described herein may be applied to any suitable joint. While the term patient is used herein, it will be appreciated that the techniques may be applied to data associated with any individual. The patient data 104 indicates a joint space between bones of the joint of interest of the patient. The patient data 104 is obtained using a parameterisation of the musculoskeletal joint of interest of the patient generated based upon three-dimensional image data in which features of the joint of interest have been identified. For example, the three-dimensional image data may be X-ray computed tomography image data and the parameterisation may be generated by fitting to the three-dimensional image data a statistical model such as an active appearance model or active shape model, which has been created based upon a training set of images of joints of interest of the same type as the joint of interest corresponding to the subject of the patient data 104. The model may be any statistical model of the variation within the training images. The statistical model may for example be generated by processing the training set of images to generate a mean model and a range of variation from the mean model of the training set. The parameterisation indicates features of the joint of interest represented in the image data and can therefore be used to identify features of the represented joint of interest. A suitable model fitting technique is described in International Patent Publication No. WO2011/098752, the contents of which are hereby incorporated by reference.

As described in further detail below, the patient data may provide a normalised joint space width for the patient. The patient data may be normalised for the sex of the patient. The patient data may additionally or alternatively be normalised based upon joint space widths associated with healthy joints of the joint of interest and may, for example, provide a z-score or other score indicating a relationship between the joint space width of the patient and typical healthy joints. The patient data may be associated with a plurality of measurements obtained from the joint of interest and may, for example, provide an average of the plurality of measurements and/or may provide an average of a particular centile of the measurements.

The computer 102 is further arranged to receive relationship data 106. The relationship data 106 is data indicating a relationship between joint space width and a property associated with cartilage of the joint of interest. While in the following reference will be made to a property indicating a likelihood of cartilage loss in the joint of interest, it will be appreciated that other properties of cartilage may be modelled using suitable relationship data that models a relationship between joint space width and the property of cartilage.

The relationship data may be generated based upon analysis of joints of interest of patients to determine both joint space width in the joints of interest of patients and data indicative of loss of cartilage in the joints of interest of the patients (or another property as discussed above). As described in further detail below, the relationship data may define a relationship between particular values of the patient data 104 indicating joint space for the joint of interest of the patient and a likelihood of cartilage loss associated with the patient data 104.

The data indicative of loss of cartilage may be generated by examining the joints of interest to determine the corresponding joint space width and loss of cartilage using any known method. In one example, the data indicative of loss of cartilage in the joint of interest may be determined based on cartilage thickness measurements obtained for each joint of interest of the patients. For example, a particular joint of interest of a patient used to generate the relationship data 106 may be classified as indicative of loss of cartilage if it is determined that a cartilage thickness of the particular joint of interest is less than a cartilage thickness of the 95^{th} percentile of a population comprising one or more corresponding joints of interest. In other words, a loss of cartilage in the joint of interest may be determined by comparing the cartilage thickness of the joint of interest with cartilage thickness data for a population. The relationship data 106 may be generated based upon joint space width data and cartilage data obtained from a population having healthy joints, for example from joints from individuals not exhibiting any indications of disease such as OA. The cartilage thickness may be a normalised value as described herein, for example normalised based upon a sex of the patient associated with each joint. The inventors have realised that cartilage thickness of a population having healthy joints when normalised only for sex falls within a surprisingly normal distribution. Given such a normal distribution, if a cartilage thickness falls outside of the 95^{th} percentile the cartilage thickness has been found to provide useful information about a likelihood of cartilage loss for a patient. The likelihood of cartilage loss may be indicative of cartilage loss that has occurred or that will occur in future, or both.

The patient data 104 is processed based upon the relationship data 106 to generate anatomical data 108. The anatomical data 108 provides a likelihood of cartilage loss in the joint of interest of the patient. The anatomical data 108 may be a discrete or continuous numerical value indicating a likelihood of cartilage loss, or may be a label indicating that the patient data 104 corresponds to a class representing a classification indicative of a likelihood of cartilage loss.

Obtaining accurate measurements for cartilage thickness can be challenging and expensive. For example, it is not generally possible to image cartilage using methods other than magnetic resonance imaging or arthrogram computed tomography, which are not typically widely available. The inventors have realised, however, that by determining a relationship between joint space width and cartilage loss, a likelihood of cartilage loss may be determined which may remove the need to image cartilage directly.

The patient data 104 may be 3-dimensional image data of a knee joint of a patient and the relationship data 106 may be data indicating a relationship between a joint space width of the knee joint of the patient and a likelihood of cartilage loss in the knee joint of the patient. The anatomical data 108 may comprise data indicating a likelihood of cartilage loss in the knee joint of the patient. In this example, the data indicating a likelihood of cartilage loss in the knee joint of the patient may be used to determine that cartilage has been lost in the knee joint and/or that cartilage will be lost in the knee joint relatively quickly compared to knees that are not indicated as having a likelihood of cartilage loss. The data indicating a likelihood of cartilage loss may indicate one or more of a plurality of properties of the patient, for example that the patient may have a degenerative joint disease or that a change in biomechanics of the knee joint has occurred. As described in further detail below, the likelihood may be associated with one or more compartments of the joint of interest. The likelihood of cartilage loss in the knee joint may provide useful information when performing a medical intervention on the knee (e.g. knee surgery). In this way, information regarding the knee joint which may not otherwise be available is made available in a probabilistic and data-driven manner, thereby contributing to an improvement in patient outcomes. In some implementations, determining a likelihood of cartilage loss may be used to inform a diagnosis of OA.

It will also be appreciated that the anatomical data 108 may be used to provide a medical intervention. For example, determining a likelihood of cartilage loss may be used to determine an appropriate medical intervention specifically targeted at addressing cartilage loss and/or any medical conditions associated thereon. For example, surgical or pharmaceutical interventions may be determined based upon the likelihood of cartilage loss.

The anatomical data 108 may be used to provide a medical intervention. For example, determining a likelihood of cartilage loss in the joint of interest of the patient allows surgical intervention that is tailored to the patient based upon the patient's joint itself. For example, a surgical plan suitable for guiding a surgeon, or for guiding automated surgical equipment, may be generated based upon the anatomical data 108. The surgical plan may, for example, provide data indicating how a joint should be modified by a surgical procedure to return the joint to a non-diseased state and/or to return the joint to a state in which it is able to function effectively. For example, a likelihood of cartilage loss in each of a plurality of compartments of the joint of interest may be determined. The likelihoods may be used to determine a status associated with each of the plurality of compartments. Based upon the likelihoods for each respective compartment, it may be determined to perform a particular surgical intervention. For example, in some implementations the anatomical data 108 may be used to determine to perform a unicondylar knee replacement or a total knee replacement.

The anatomical data 108 may additionally or alternatively be used for non-surgical interventions such as for analysis of efficacy of drugs, physiotherapy or any other medical intervention that may be used to mitigate a condition. The condition may be osteoarthritis. The anatomical data 108 may comprise data suitable for generation of a prosthesis for the musculoskeletal joint of interest of the patient.

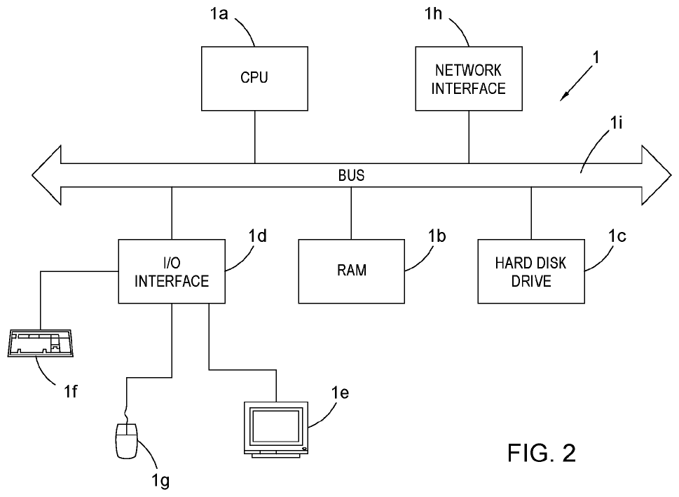

FIG. 2 shows a computer 1, the computer 1 being the computer 102 of FIG. 1 described in further detail. It can be seen that the computer 1 comprises a CPU 1a which is configured to read and execute instructions stored in a volatile memory 1b which takes the form of a random access memory. The volatile memory 1b stores instructions for execution by the CPU 1a and data used by those instructions. For example, in use, the relationship data 106 and patient data 104 FIG. 1 may be stored in the volatile memory 1b.

The computer 1 further comprises non-volatile storage in the form of a hard disc drive 1c. The data generated by the relationship data 106 and the patient data 104 may be stored on the hard disc drive 1c. The computer 1 further comprises an I/O interface 1d to which is connected peripheral devices used in connection with the computer 1. More particularly, a display 1e is configured so as to display output from the computer 1. The display 1e may, for example, display a representation of the patient data 104, relationship data 106, and/or the anatomical data 108. Input devices are also connected to the I/O interface 1d. Such input devices may include a keyboard 1f and a mouse 1g which allow user interaction with the computer 1. A network interface 1h allows the computer 1 to be connected to an appropriate computer network so as to receive and transmit data from and to other computing devices. The CPU 1a, volatile memory 1b, hard disc drive 1c, I/O interface 1d, and network interface 1h, are connected together by a bus 1i.

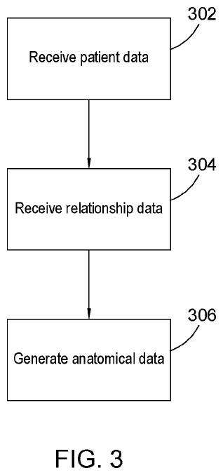

FIG. 3 shows processing carried out to generate the anatomical data 108. The processing may occur on the computer 1.

At step 302, the patient data 104 is received. As described herein, the patient data 104 indicates joint space width of a joint of interest of the patient. In one example, the patient data 104 comprises joint space width measurements as described herein. Alternatively, the patient data 104 may comprise one or more normalized numerical values indicative of joint space width. For example, the one or more normalized numerical values may be one or more z-scores.

At step 304, the relationship data 106 is received. As described herein, the relationship data 106 is data indicating a relationship between joint space width and likelihood of cartilage loss in the joint of interest. In one example, the relationship data 106 may comprise one or more ranges of values each corresponding to a different likelihood of cartilage loss.

At step 306, the patient data 104 and the relationship data 106 are processed to generate the anatomical data 108 associated with the musculoskeletal joint of interest of the patient. The processing may comprise comparing the patient data 104 with the relationship data 106. For example, as described in further detail below, different values or ranges of values in the relationship data may correspond to particular likelihoods of cartilage loss. For example, a first range of values in the relationship data 106 may correspond to a low likelihood of cartilage loss, and a second range of values in the relationship data 106 may correspond to a high likelihood of cartilage loss. In the case that the patient data 104 (i.e. joint space width) is within the second range and not within the first range, this may indicate that the joint of interest has a high likelihood of cartilage loss.

The relationship data 106 provides a model that allows cartilage loss to be predicted from joint space width data for a patient. The relationship data 106 may be determined from analysis of image data associated with a plurality of patients in which cartilage loss and joint space width can both be determined as described above. The inventors have surprisingly realised that the relationship between joint space width across a population and cartilage loss across the same population can be used to predict cartilage loss for a particular patient based upon the particular patient's joint space width. While it is generally understood that joint space narrowing for a particular patient may be indicative of cartilage loss in the joint, a large number of factors are generally understood to contribute to joint space width and cartilage loss in a complex way that is not well understood. Joint space width has therefore previously been used as one of a number of indicators in determining development of diseases such as osteoarthritis. The inventors have realised, however, that it is possible to determine relationship data 106 as described herein that allows accurate prediction of likelihood of cartilage loss without requiring either expensive imaging modalities or computationally expensive image processing.

The anatomical data 108 therefore can be used to predict cartilage loss in a joint of interest. The anatomical data can therefore be used to improve determination of whether to provide a medical intervention, for example performing a total knee arthroplasty.

As described above, the relationship data 106 may be determined from analysis of image data associated with a plurality of patients in which cartilage loss and joint space width can both be determined. Joint space width indicates distance between two bones of a joint. Processing of image data to determine joint space width is described in further detail below. While identifying bone in an image of a joint is relatively straightforward, obtaining accurate data indicating cartilage is generally more challenging. The image data used to determine the relationship data 106 may, for example, be obtained using magnetic resonance imaging (MRI). The image data may be manually reviewed and labelled or may be processed using an automated method based on, for example, shape modelling and deep learning in order to determine cartilage thickness which allows cartilage to be accurately identified. An example technique for measuring cartilage thickness is described in “Precision, Reliability, and Responsiveness of a Novel Automated Quantification Tool for Cartilage Thickness: Data from the Osteoarthritis Initiative”, Bowes et. al., The Journal of Rheumatology, February 2020, 47 (2) 282-289, the contents of which is incorporated herein by reference.

Joint space width in a joint of interest may be determined in any convenient way. In general terms joint space width associated with patient data and joint space width associated with the image data associated with a plurality of patients used to generate relationship data is determined in a corresponding manner in order to provide consistency between the patient data and the relationship data. One way of determining joint space width in a joint of interest will now be described. While in the following joint space width is described with respect to a knee joint, it will be appreciated that the technique described can also be applied in the context of other joints of interest.

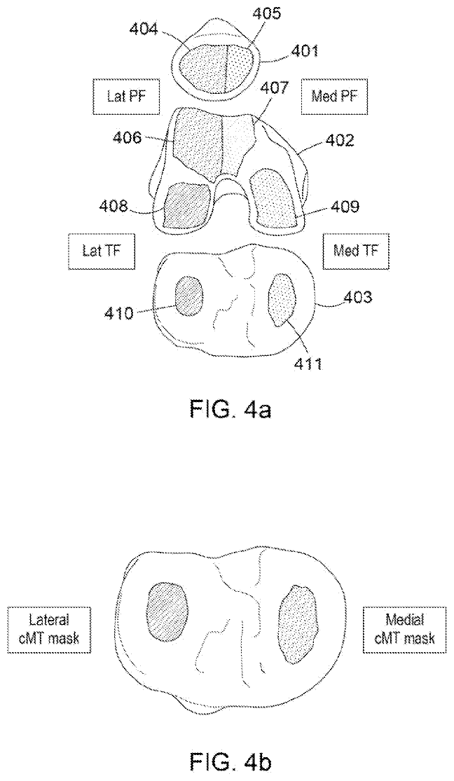

Referring to FIG. 4a, three-dimensional image data of a patella 401, a femur 402 and a tibia 403 of a knee joint are shown. Patches 404 to 411 are each associated with respective surfaces of bones of the knee joint that may be used to determine joint space width of different parts of the knee joint. Each patch 404 to 411 may comprise a plurality of points of interest associated with the surface of the respective bone surface. The plurality of points of interest may be determined by determining a segmentation of the bone surface and fitting a mask to the segmentation. The segmentation may be obtained in any convenient way, for example using the model fitting technique described in International Patent Publication No. WO2011/098752. An example mask showing a plurality of lines on which multiple points of interest are located is shown in FIG. 4b, although it will be appreciated that the mask may take any convenient form and different points of interest may be used to those shown in FIG. 4b.

A distance may be determined between one or more patches of the plurality of patches 404 to 411. As shown in FIG. 4a, patches 404 and 406 are associated with the lateral patellofemoral compartment, patches 405 and 407 are associated with the medial patellofemoral compartment, patches 408 and 410 are associated with the lateral tibiofemoral compartment and patches 409 and 411 are associated with the medial tibiofemoral compartment. A joint space width for one or more of the lateral patellofemoral compartment, medial patellofemoral compartment, lateral tibiofemoral compartment and medial tibiofemoral compartment can be determined based upon a distance in three-dimensional space between the respective patches associated with a particular one of the compartments. In particular, each pair of patches associated with a particular compartment may comprise a plurality of points, with each point of the plurality of points in a first of the pair of patches being associated with a respective point in of the plurality of points in a second of the pair of patches. A distance may be determined between each of the associated respective points to provide a plurality of distances between associated respective points for the particular compartment. The plurality of determined distances may be combined to provide an indication of joint space width for the particular compartment (e.g. for the lateral patellofemoral compartment, etc.). In one example, the knee joint may be under compression when the three-dimensional image data depicted in FIGS. 4a and 4b is generated. For example, the patient may be standing erect and/or weight bearing, thereby applying compressive force to the knee joint. In another example, the joint of interest may be flexed within a particular range of flexion. In this example, the range of flexion may be about 15 degrees of flexion or greater, for example between 15 and 20 degrees of flexion. In other words, the patches, the distances, and the indication of joint space width may all be determined when the joint of interest is under compression. In this way, any mechanical or positional factors which may affect a true indication of joint space width for the particular compartment may be mitigated.

The plurality of distances may be combined in any convenient way to provide the indication of joint space width for the particular compartment. For example, an average value may be determined indicating an average of the plurality of points. The average may be a mean, median or mode value. Additionally, or alternatively, the plurality of distances may be combined by obtaining a predetermined centile of distance values. The predetermine centile of distance values may be, for example, a smallest ten percent of distances, a smallest twenty percent of distances or the like. The predetermined centile of distance values may be combined, for example by taking an average as described above. Using a predetermined centile of distance values has been found to provide improvements in particular compartments. For example, cartilage loss in the patellofemoral compartment has been determined for some measurement techniques to only occur across a small portion of the patch in some patients. By using a predetermined centile of distance values in such compartments, therefore, joint space information can be obtained that represents damage in the compartment of interest in an improved way.

In some implementations, cartilage loss determination in patellofemoral compartments can in some embodiments be improved by imaging the knee under predetermined conditions. For example, by imaging a knee joint with a flexion angle of about 15 degrees or greater has been determined to improve determination of cartilage in patellofemoral compartments. When a knee is imaged with little or no flexion, the patella is able to disengage from the femur meaning that imaging cartilage between the patella and femur is more challenging. As described above, it has been determined that using a smallest percentile of measurements can mitigate such challenges. In some implementations, the particular percentile of measurements used can be determined based upon how the knee joint is imaged. In some implementations the joint can be processed using a shape model to determine whether the joint can be processed to determine cartilage by determining a location of the patella relative to the femur. In some implementations the joint may be imaged standing. The joint may be imaged with a predetermined angle of flexion applied to the joint, for example using a knee brace. In some implementations the predetermined conditions are applied to joints used for both generation of relationship data and patient data to provide consistency between the model and input data.

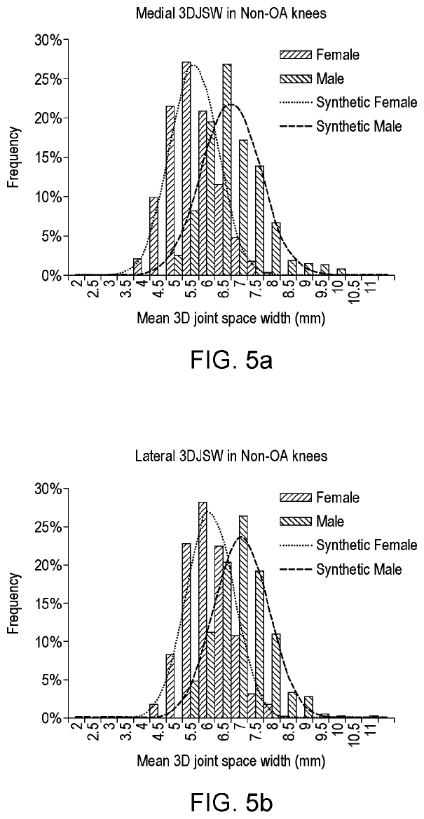

The indication of joint space width for the particular joint may further comprise normalised values. For example, joint space widths determined as described above with reference to FIG. 4 may be further normalised to provide the patient data 104 and/or to provide joint space width data used to generate the relationship data 106. In some implementations the joint space widths determined as described above are normalised with respect to sex of a patient. FIGS. 5a and 5b respectively show plots of joint space width for medial and lateral tibiofemoral compartments across a population, with joints of interest associated with males and females plotted separately in each of FIGS. 5a and 5b. As can be seen in each of FIGS. 5a and 5b, male joint space width is greater than female joint space width in each of the medial and lateral joints. Joint space width data for the patient and/or for each of the population of joints used to generate relationship may be normalised by adjusting the joint space width for the sex of the patient associated with the joint.

In some implementations, the data is additionally or alternatively normalised based upon healthy joint space width. For example, the joint space width measurements described above with reference to FIG. 4 may be normalised by determining variation of the joint space width from an average healthy joint space width. The normalisation based upon healthy joint space width may, for example, generate a z-score for the joint space width based upon a standard deviation and a mean joint space width determined across a population of healthy joints, for example joints in which no cartilage loss is observed.

The population of healthy joints may be determined for male and female populations and the normalisation based upon healthy joint space width may therefore provide a normalisation for both healthy joint space width and sex. In some implementations, only two populations of healthy joints may be used such that the normalisation is based upon healthy joint space width and sex only. That is, in some implementation no other properties of the patient may be taken into account in the normalisation. The inventors have realised that sex is the only factor affecting healthy cartilage thickness that is sufficiently significant to require correction. Therefore, the inventors have surprisingly found that it is not necessary to normalise for properties other than sex of the patient in order to provide a model that is predictive of likelihood of cartilage loss based upon joint space width. In this way, the accuracy of predicting a likelihood of cartilage loss may be improved.

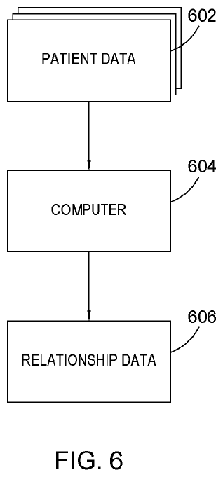

FIG. 6 schematically shows generation of the relationship data 106. As shown in FIG. 6, patient data 602 is received by computer 604 (which may be the same computer of FIGS. 1 and 2 or may be a different computer). The patient data 602 may comprise joint space width data associated with a plurality of joints of interest and corresponding cartilage loss data indicating presence or absence of cartilage loss for each of the plurality of joints of interest. The computer is configured to generate as output relationship data 606. The relationship data 606 may be the relationship data 106 of FIG. 1. The relationship data 606 indicates a relationship between joint space width and cartilage loss. The patient data 602 may comprise joint space width data obtained as described herein with reference to FIGS. 4a and 4b and may, for example, be normalised joint space width data and may be associated with a particular population such as a population of a single sex.

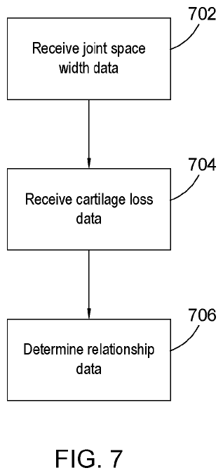

FIG. 7 depicts a flow diagram of a method used to generate the relationship data 106. At step 702, joint space width data is received. The joint space width data comprises an indication of joint space width for one or more joints of interest in a population. The joint space width data may comprise one or more joint space width values each corresponding to different joint space widths (e.g. lateral and medial joint space widths) for the respective one of the one or more joints of interest. In one example, the one or more joints of interest are the same type of joint. In one example, the population is discriminated based upon sex.

At step 704, cartilage loss data is received. The cartilage loss data comprises an indication of cartilage loss for each of the one or more joints of interest in the population. The indication of cartilage loss may be a continuous or discrete numerical value, or may be a label. For example, the continuous or discrete numerical value, or label, may indicate that a corresponding joint space width is indicative of cartilage loss.

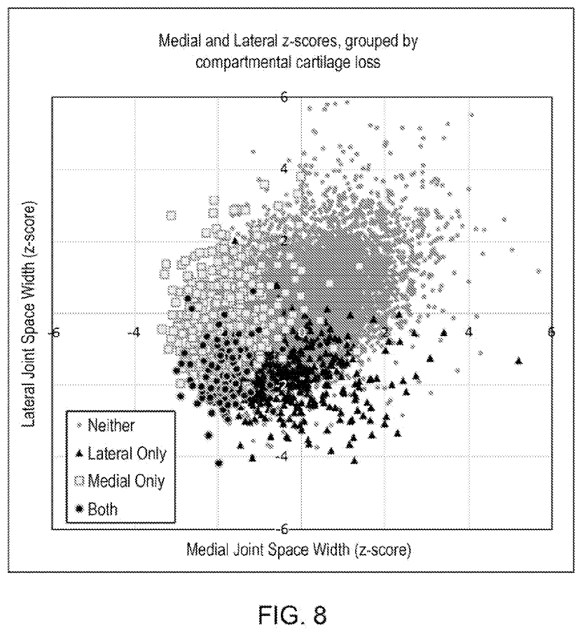

At step 706, the relationship data 106 is determined. The relationship data 106 may be determined based upon the received joint space width data and the received cartilage loss data. The relationship data 106 may indicate a likelihood of cartilage loss based upon a joint space width for a specific joint of interest. For example, the relationship data 106 may be determined using a statistical method. In another example, the relationship data 106 may be determined by segmenting joint space width values based upon the corresponding cartilage loss data, as described below with reference to FIG. 8. In another example, the relationship data 106 may be determined using a machine learning algorithm configured to learn a relationship between joint space width values and cartilage loss. In some implementations, the relationship data may represent a relationship between joint space widths for a plurality of joints of interest and cartilage loss.

FIG. 8 depicts patient data 802 suitable for determining relationship data 808. The patient data comprises, for each of a plurality of patients, a normalised medial tibiofemoral compartment space width and a normalised lateral tibioofemoral joint space width. In FIG. 8, each of the x-axis and y-axis represents a z-score indicating variation of the respective joint space widths relative to an average joint space width of a population of healthy joints associated with patients of the same sex as the respective joint space widths. Because the joint space widths are normalised in this way, data associated with both male and female patients may be considered in combination. Colours of points in FIG. 8 associated with particular patients indicate whether the particular patient associated with the point has been determined to have cartilage loss in either the medial joint space, the lateral joint space, both medial and lateral joint spaces or neither.

As can be seen in FIG. 8, patients with cartilage loss in particular joints are clustered in particular regions associated with particular z-scores. Based upon the distribution of patients with cartilage loss and without cartilage loss associated with particular z-scores it is possible to determine for each z-score associated with particular joint space widths a likelihood that a patient has cartilage loss, and in FIG. 8 specifically, a likelihood that a patient has cartilage loss in the medial compartment, lateral compartment, or both. Determining a likelihood for individual compartments allows improved medical interventions to be made. For example, if cartilage loss is likely in the medial compartment only, this may inform a surgeon to perform surgery on the medial compartment only, and not replace the entire knee joint.

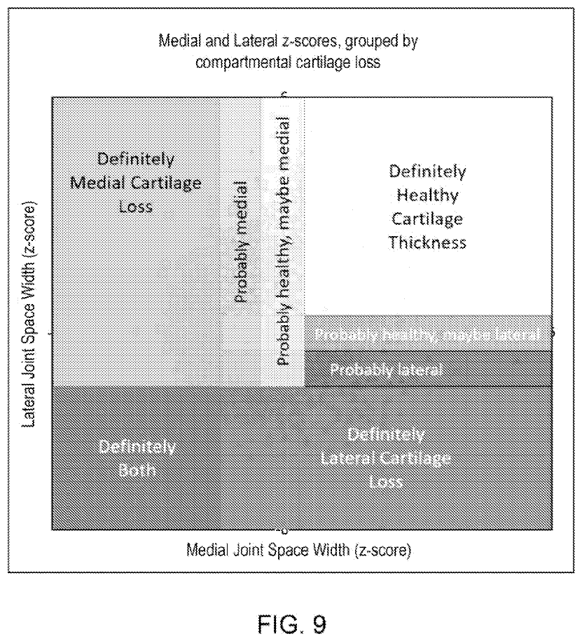

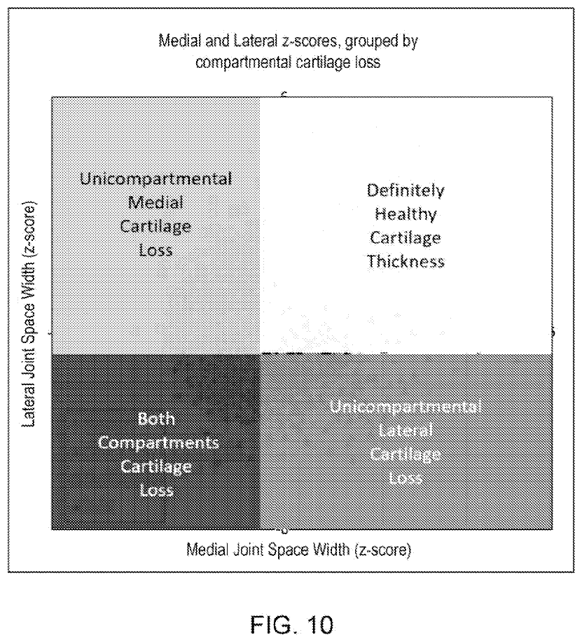

FIG. 9 illustrates a plurality of likelihoods of cartilage loss associated with particular z-scores according to the data illustrated in FIG. 8 although it will be appreciated that the likelihood that a patient has cartilage loss can be provided in any convenient form. For example, in some implementations the likelihood is provided as an output suitable for use by clinicians as shown in FIG. 10 in which the z-score is represented together with a heat map indicating likelihood of cartilage loss. It will be appreciated, however, that while FIGS. 9 and 10 each provide a representation of likelihoods that is suitable for clinicians, the patient data provides a value between 0 and 1 for each z-score or combination of z-scores.

While FIGS. 8 to 10 are based upon two joints of interest, it will be appreciated that other numbers of joints of interest can be modelled. For example, each of the lateral and medial tibiofemoral compartments and the lateral and medial patellofemoral compartments may be modelled individually or in combination to provide a likelihood of cartilage loss based upon a corresponding combination of one or more joint space widths for the joints.

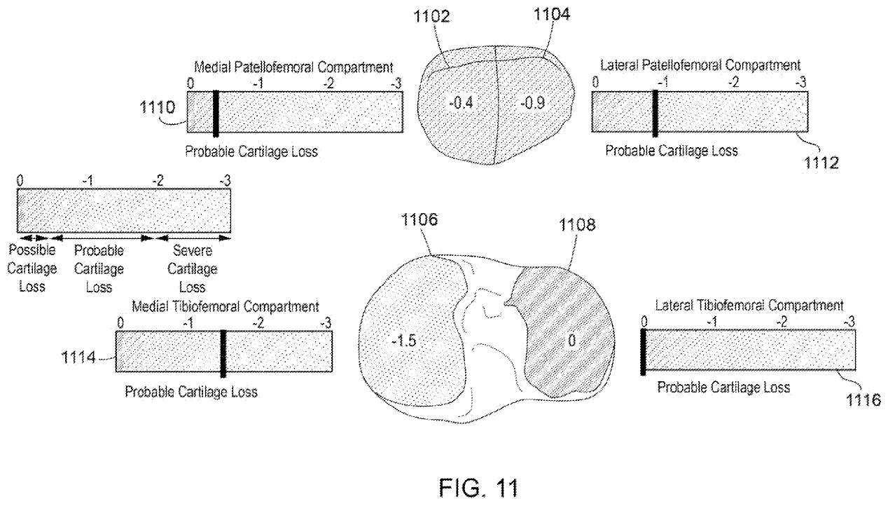

FIG. 11 depicts four compartments of a knee joint. In particular, a medial patellofemoral compartment 1102, a lateral patellofemoral compartment 1104, a medial tibiofemoral compartment 1106, and a lateral tibiofemoral compartment 1108 are shown. For each compartment of the knee joint, a z-score is overlaid indicating the likelihood of cartilage loss. For example, the lateral tibiofemoral compartment 1108 has a z-score of 0 overlaid, indicating possible cartilage loss in this compartment. In another example, the lateral patellofemoral compartment 1104 has a z-score of −0.9 overlaid, indicating probable cartilage loss in this compartment. A z-score of 0 indicates that a cartilage thickness in a given compartment is equivalent to the mean cartilage thickness for a population of knee joints. A z-score of −1 indicates that a cartilage thickness in a given compartment is 1 standard deviation less than the mean cartilage thickness for the population of knee joints. A z-score of 1 indicates that a cartilage thickness in a given compartment is 1 standard deviation greater than the mean cartilage thickness for the population of knee joints. In this case, the knee joint comprising the medial patellofemoral compartment 1102, the lateral patellofemoral compartment 1104, the medial tibiofemoral compartment 1106, and the lateral tibiofemoral compartment 1108 has probable cartilage loss in three out of four of its compartments. It will be readily appreciated that various approaches for indicating a likelihood of cartilage loss may be envisaged. For example, FIG. 11 depicts z-scores overlaid on knee joints, with four corresponding z-score ranges 1110 1112 1114 1116, each used to determine the likelihood of cartilage loss their respective knee joint compartment. Other scoring metrics may be envisaged, such as a percentage or percentile score indicating one of a plurality of percentile ranges, a T-score on a T-score range, a cartilage thickness measure on a cartilage thickness measurement range, or any other suitable scoring system. As described herein, this may not only indicate that the knee joint has lost more than a usual amount of cartilage, but that this knee joint may be likely to continue to lose cartilage at a higher than usual rate.

To determine a likelihood of cartilage loss for a new patient, lateral and medial joint space width data determined for the patient can be input to the model (i.e. the patient data 104 of FIG. 1) to determine a likelihood of cartilage loss. For example, where a medial and lateral joint is modelled as shown in FIGS. 8 to 10, the medial and lateral joint space widths can be input to directly determine a likelihood. As described above, the likelihood may be a probabilistic value between 0 and 1 where a value close to 0 indicates that the probability of the patient having cartilage loss is low and a value close to 1 indicates that the probability of the patient having cartilage loss is high.

The methods and systems described herein may be used to determine a likelihood of cartilage loss, for example, in the event that a patient reports experiencing joint pain to a clinician. It may be determined that cartilage loss is likely or unlikely to have occurred based upon a measurement of joint space width. This measurement of joint space width may be obtained, as described herein, more easily than by directly and accurately measuring cartilage thickness in the joint. It may be the case that clinicians perform medical interventions in light of a perceived cartilage loss when cartilage loss has not occurred due. Cartilage loss may have been perceived based upon an observed joint space width narrowing. Joint space width narrowing may occur, for example, if the meniscus in the compartment is no longer functioning correctly. In this case, no cartilage may have been lost, but the joint space width may still be reduced, thereby resulting in an incorrect assumption that cartilage loss has occurred. The methods and systems described herein overcome this problem by determining a likelihood of cartilage loss.

While reference in the above is generally made to a likelihood of cartilage loss, as indicated above, other properties may be modelled and determined. For example, the patient data used to train the model may indicate a structural quality associated with cartilage. The structural quality associated with cartilage may be determined using T2 values from MRI images. Additionally or alternatively, the property may indicate a likelihood that a point, or a group of points, are completely denuded indicating that bone is in contact with bone at an associated imaged point. Additionally, or alternatively, the property may indicate a likelihood of future cartilage loss at a point in time in the future, utilising a training model which contains data for multiple points in time. The relationship data may be processed with respect to joint space data and the output may be indicative of the property of cartilage associated with the patient data used to train the model.

The methods and systems described herein have been illustrated with reference to generating and/or determining a likelihood of cartilage loss. However, it will be readily appreciated that any other property of cartilage in the joint of interest of the patient may be determined. For example, other properties of cartilage may be cartilage status and/or condition.

1. A method of determining a property of cartilage in a joint of interest of a patient, the method comprising:
receiving data indicating joint space width of the joint of interest;
receiving data indicating a relationship between joint space width and a property of cartilage in the joint of interest, wherein the data indicating a relationship between joint space width and the property of cartilage in the joint of interest is based upon measured cartilage in a population of joints of interest; and
determining the property of cartilage in the joint of interest based upon the data indicating joint space width of the joint of interest and the data indicating a relationship between joint space width and property of cartilage in the joint of interest.

2. A method according to claim 1, wherein the property of cartilage is a likelihood of cartilage loss and/or a structural quality of cartilage.

3. A method according to claim 1, wherein the data indicating joint space width of the joint of interest comprises one or more normalized values associated with the joint space width.

4. A method according to claim 3, wherein the one or more normalized values are determined based upon the sex of the patient.

5. A method according to claim 3, wherein the one or more normalized values are determined based upon healthy joint space width data indicating healthy joint space width for one or more healthy joints.

6. A method according to claim 1, wherein each patient associated with the one or more healthy joints do not exhibit symptoms associated with a disease affecting cartilage.

7. A method according to claim 3, wherein the one or more normalized values indicate a variation from one or more average values associated with one or more healthy joints corresponding to the joint of interest.

8. A method according to claim 1, wherein the data indicating joint space width is determined based upon one or more images of the joint of interest.

9. A method according to claim 8, wherein the data indicating joint space width of the joint of interest is determined based upon image processing that does not determine cartilage.

10. A method according to claim 8, wherein the one or more images are generated by computed tomography and/or magnetic resonance imaging.

11. A method according to claim 3, wherein the data indicating joint space width of the joint of interest is determined from first image data and the data indicating a relationship between joint space width and property of cartilage in the joint of interest is determined from second image data different to the first image data.

12. A method according to claim 3, wherein the data indicating a relationship between joint space width and property of cartilage in the joint of interest is obtained based upon data obtained from a plurality of joints of interest in which joint space width and cartilage loss in the joint of interest are known.

13. A method according to claim 1, wherein the joint of interest comprises a lateral compartment and a medial compartment.

14. A method according to claim 13, wherein the data indicating joint space width comprises lateral joint space width data corresponding to the lateral compartment and medial joint space width data corresponding to the medial compartment.

15. A method according to claim 1, further comprising:
determining, based upon the property of cartilage, whether or not the joint of interest is affected by osteoarthritis.

16. A method according to claim 1, wherein the data indicating joint space width is determined based upon a plurality of measurements between predetermined points of the joint of interest.

17-21. (canceled)

22. A method according to claim 1, wherein the joint space width is generated based upon joints of interest under compression and/or load bearing.

23. A computer program comprising computer readable instructions configured to cause a computer to:
receive data indicating joint space width of a joint of interest of a patient;
receive data indicating a relationship between joint space width and a property of cartilage in the joint of interest, wherein the data indicating a relationship between joint space width and the property of cartilage in the joint of interest is based upon measured cartilage in a population of joints of interest; and
determine the property of cartilage in the joint of interest based upon the data indicating joint space width of the joint of interest and the data indicating a relationship between joint space width and property of cartilage in the joint of interest.

24. A computer readable medium carrying a computer program according to claim 22.

25. A computer apparatus for determining a property of cartilage in a joint of interest of a patient comprising:
a memory storing processor readable instructions; and
a processor arranged to read and execute instructions stored in said memory;
wherein said processor readable instructions comprise instructions arranged to control the computer to:
receive data indicating joint space width of the joint of interest;
receive data indicating a relationship between joint space width and a property of cartilage in the joint of interest, wherein the data indicating a relationship between joint space width and the property of cartilage in the joint of interest is based upon measured cartilage in a population of joints of interest; and
determine the property of cartilage in the joint of interest based upon the data indicating joint space width of the joint of interest and the data indicating a relationship between joint space width and property of cartilage in the joint of interest.
